(19) United States
(12) Patent Application Publication
(10) Pub. No.: US 2020/0265923 A1
(43) Pub. Date: Aug. 20, 2020

(54) Efficient Seeding For Read Alignment

(71) Applicant: THE REGENTS OF THE UNIVERSITY OF MICHIGAN, Ann Arbor, MI (US)

(72) Inventors: Arun SUBRAMANIYAN, Ann Arbor, MI (US); Satish NARAYANASAMY, Ann Arbor, MI (US); Reetuparna DAS, Ann Arbor, MI (US); David T. BLAAUW, Ann Arbor, MI (US)

(21) Appl. No.: 16/749,139

(22) Filed: Jan. 22, 2020

Related U.S. Application Data
(60) Provisional application No. 62/795,188, filed on Jan. 22, 2019.

(51) Int. Cl. G16B 30/10; G06F 16/22

(52) U.S. Cl. CPC G16B 30/10; G06F 16/2282; G06F 16/2246

(57) ABSTRACT

Read alignment is a time-consuming step in genome sequencing analysis. The most widely used software for read alignment, BWA-MEM and BWA-MEM2 are based on the seed-and-extend paradigm for read alignment. The seeding step of read alignment is a major bottleneck contributing ˜38% of the overall execution time in BWA-MEM2 when aligning whole human genome. This is because BWA-MEM2 uses a compressed index structure called the FMD-Index, which results in high bandwidth requirements, primarily due to its character-by-character processing of reads. To address these challenges, a novel seeding data structure is presented along with a custom accelerator architecture for seeding.

### CROSS-REFERENCE TO RELATED APPLICATIONS

This application claims the benefit of U.S. Provisional Application No. 62/795,188, filed on Jan. 22, 2019. The entire disclosure of the above application is incorporated herein by reference.

### FIELD

The present disclosure relates to an efficient seeding for read alignment of genome data.

### BACKGROUND

Genomics can transform precision health over the next decade, by providing solutions ranging from early cancer detection to customized drug therapies and treating rare genetic disorders. A genome is essentially a long string (6 Giga bp for a human genome) of DNA base-pairs (bp) A, G, C, and T. During primary analysis, a sequencing instrument splits a DNA into billions of short (˜100 bp) strings called reads. Secondary analysis aligns the reads to a reference genome and determines genetic variants in the analyzed genome compared to the reference. This work focuses on aligning short reads, since more than 70% of the direct-to-consumer (DTC) genomics market is currently serviced by Illumina short read sequencers.

Read alignment is one of the major compute bottlenecks in secondary analysis. Every read needs to be aligned to a position in the reference genome. Naively aligning by matching a string to every possible position in the reference genome is computationally intractable. Read aligners solve this using seeding. Seeding finds a set of candidate locations (hits) in the reference genome where a read can potentially align. Hits for a read are determined by finding exact matches for its substrings (seeds) in the reference. The seed extension phase then uses approximate string matching to select the hit with the best score as the read's alignment position.

In addition to read alignment, seeding is also an important kernel in several other sequencing applications: metagenomics classification (e.g., Centrifuge), de-novo assembly, read error correction, etc.

Several studies in the past have designed efficient accelerator solutions for seed-extension. However, efficient accelerators for seeding are lacking despite being a performance bottleneck in commonly used read aligners. For instance, seeding contributes ˜38% to the overall run time of state-of-the-art read aligner BWA-MEM2. This disclosure focuses on seeding in BWA-MEM2, as it is the fastest available implementation of BWA-MEM, which is recommended as industry standard in the Broad Institute's best practices genomics pipeline.

The primary performance bottleneck in seeding is memory bandwidth. This is because both BWA-MEM and BWA-MEM2 use a compressed index structure called the FMD-Index. When compared to BWA-MEM, BWA-MEM2 uses a lower compression factor for the index to reduce memory bandwidth requirements, but because of iterative processing of each base-pair in a read it still has high bandwidth requirements. Experiments on real whole human genome data show that each short read (with 101 base-pairs or 37.5 B) requires an average of 61.3 KB of data from main memory to seed. That is about 45 TB of data for the whole genome. Furthermore, each of the index accesses tends to touch a different part of the 42 GB index data structure, and exhibits little spatial or temporal locality.

The memory bandwidth bottleneck can be understood using the roofline plot shown in FIG. 1A. The roofline is the maximum performance achievable on a system with 136 GB/s peak DRAM bandwidth for a given data efficiency (data fetched from memory per analyzed read). BWA-MEM2 on an AWS CPU instance with 72 cores utilizes about half of the peak memory bandwidth (blue triangle). Hence, even an infinitely fast and parallel FMD-index hardware accelerator cannot achieve more than 2.1× speedup over the CPU instance due to its memory bandwidth bottleneck (blue circle) unless data requirements of the algorithm are reduced. Existing hardware accelerators for seeding have used FMD-Index and are thus all limited by this upper limit.

To address these challenges, this disclosure presents a novel data structure with 4.3× higher data efficiency than BWA-MEM2 and an accompanying custom accelerator architecture for seeding.

This section provides background information related to the present disclosure which is not necessarily prior art.

### SUMMARY

This section provides a general summary of the disclosure, and is not a comprehensive disclosure of its full scope or all of its features.

In one aspect, a computer-implemented method is presented for identifying a match between an input string and a portion of a reference genome sequence. To enable the alignment process, an improved seeding data structure is constructed as follows: building an index table for the reference genome sequence in a computer memory, where each entry in the index table represents a k-mer, the k-mer is comprised of nucleotides and the index table contains all possible permutations for the k-mer; for each entry in the index table, recording a presence indicator that indicates if the k-mer exists in the reference genome sequence; and, for each entry in the index table that exists in the reference genome sequence, constructing a tree for a given entry of the index table in a secondary data structure of the computer memory, where the given entry of the index table includes a pointer to the tree in the secondary data structure and the tree represents suffixes to the given entry as found in the reference genome sequence.

Once constructed, the seeding data structure can be used to search for maximal exact matches in the reference genome sequence. The method includes: extracting a read from a biological sample; receiving a k-mer from the read; retrieving an entry from an index table using the k-mer, where the index table contains an entry for each possible permutation of the k-mer and the entry includes a pointer to a tree in a secondary data structure; retrieving the tree for the k-mer from the secondary data structure using the pointer, where the tree represents suffixes to the entry as found in the reference genome sequence; traversing branches of the tree to identify matches between strings in the read and strings found in the reference genome sequence; and reporting matches as maximal exact matches when number of characters in matched strings exceeds a threshold. The branches of the tree are traversed by comparing characters in the read that follow the k-mer to suffixes represented by the tree.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1A is a graph showing how seeding data structure improves bandwidth efficiency of FMD-Index based seeding allowing for both software and hardware acceleration of BWA-MEM2.

FIG. 1B is a graph showing the trade-off between index size and data required for seeding.

FIG. 2A is a diagram illustrating an example of super-maximal exact matches.

FIG. 2B is a diagram illustrating forward and backward searching to identify super-maximal exact matches.

FIG. 3 is a diagram illustrating a proposed data structure for efficient seeding for read alignment of genome data;

FIG. 4 is a flowchart providing an overview of a method for constructing a seeding data structure;

FIGS. 5A and 5B are flowcharts depicting building an index table and a radix tree for the seeding data structure, respectively;

FIG. 6 is a flowchart showing a method of searching for MEMs using the seeding data structure.

FIG. 7 is a diagram showing merging of radix-trees by adding prefix data at the leaf nodes allowing the seeding data structure to leverage prefix information to perform multiple MEM searches in a single tree traversal.

FIG. 8 is a diagram showing the steps to be performed to leverage k-mer reuse.

FIG. 9 is a diagram depicting a cache-friendly tiled data-layout for the seeding data structure.

FIG. 10 is a graph showing skewed hit distribution for k-mers, where few k-mers with a large number of hits are represented with an index table.

FIG. 11 is a diagram showing pruning wasteful backward searches by performing backward searches in right-to-left order.

FIGS. 12A and 12B are a diagram depicting indexing of both forward and reverse complemented reference genome to enable bidirectional search.

FIG. 13 is a diagram showing an example seeding processor architecture.

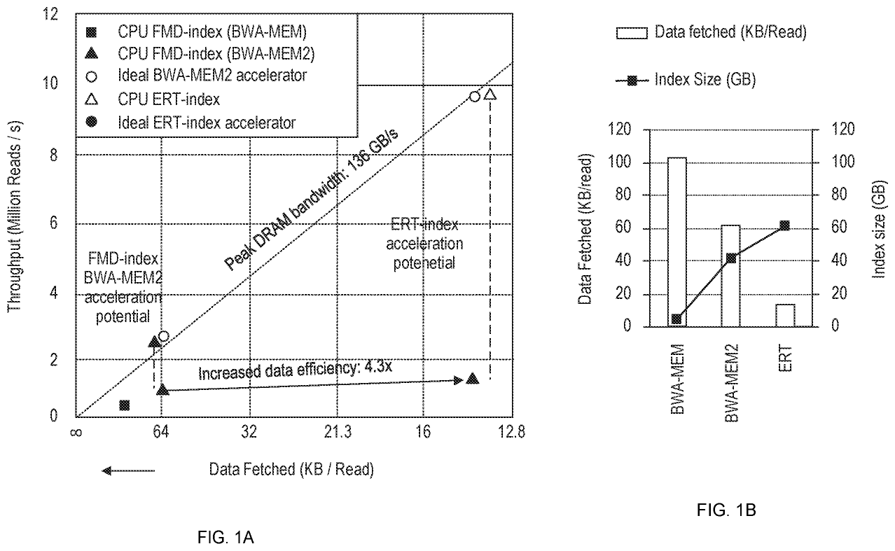

Corresponding reference numerals indicate corresponding parts throughout the several views of the drawings.

### DETAILED DESCRIPTION

Example embodiments will now be described more fully with reference to the accompanying drawings.

Seeding identifies the locations in the reference genome where a possible alignment could exist for a given read. It greatly reduces the computation required during seed extension, and is important for end-to-end read alignment performance. Seeding constitutes 38% to the overall execution time of BWA-MEM2 as measured on the whole human genome consisting of 787,265,109 reads of 101 bit length. Seed extension is lesser, 31%.

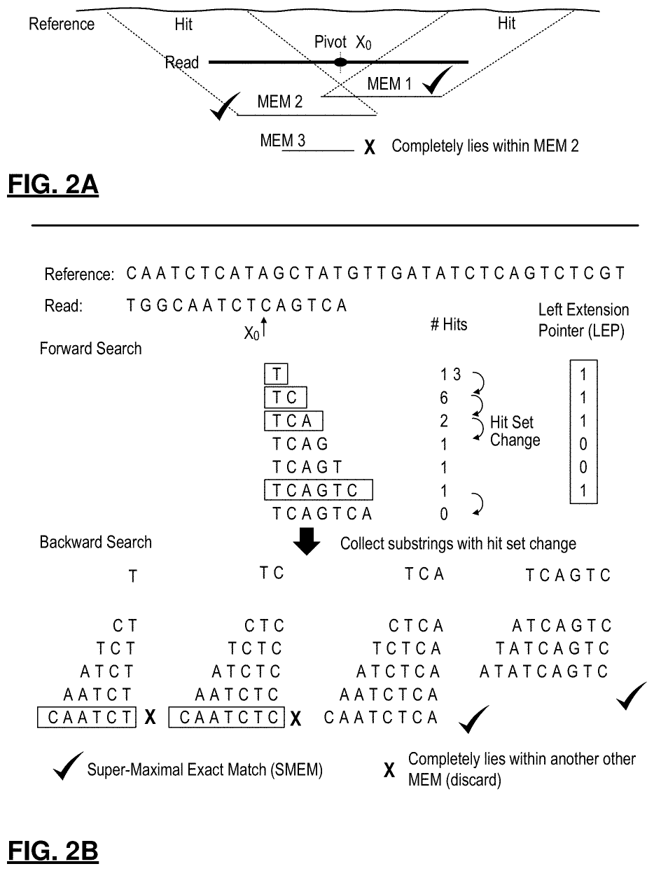

The seeding algorithm in BWA-MEM2 is based on identifying substrings that have super-maximal exact matches (SMEMs) with the reference genome as seen in FIG. 2A. A maximal exact match (MEM) is an exact match that cannot be extended in either direction in the read. An SMEM is a maximal length match (MEM) that is not fully contained in any other MEM. FIG. 2B shows the steps involved in determining SMEMs for a sample read and reference pair.

SMEMs are identified in two steps: (1) forward search and (2) backward search. For a given query position in the read (e.g., pivot xₒ in FIG. 2), subsequent base pairs to its right are looked up one at a time in a reference index to find the longest exact match in the forward direction. During this step, all the positions in the read where there is a change in the set of candidate reference locations (hits) are marked (left extension points (LEP) in FIG. 2). Only these positions are used as the starting query positions to identify MEMs that extend in the backward direction. Other positions are guaranteed to produce MEMs that are contained within those identified from LEP.

For each query position identified in the previous step, subsequent bases to its left are looked up one at a time to find the longest exact match in the backward direction. After this process, SMEMs are identified by discarding MEMs fully contained in other longer matches. The locations of these SMEMs in the reference genome (hits) are then determined and passed on to the seed-extension stage. SMEMs obtained during seeding are assumed to be part of the final alignment.

Like BWA-MEM, BWA-MEM2 also uses two other seeding heuristics to produce highly accurate seeds. The first heuristic known as reseeding breaks down long SMEMs (>28 bp) that have every few hits (<10) in the reference genome into shorter substrings with greater number of hits. The second heuristic based on the LAST aligner further identifies disjoint seeds in the read using forward search. Use of disjoint seeds reduces the probability that a read is mismapped due to sequencing errors.

To identify SMEMs and their locations in the reference genome, both BWA-MEM and BWA-MEM2 use a compressed data structure called the FMD-index which is built using both strands of DNA (˜6 billion characters for the human genome). The FMD-index allows the lookup of query Q of length N in reference R using approximately O(N) memory operations. The FMD-index is utilized for all the three steps of seeding described earlier (SMEM generation, reseeding, and LAST).

BWA-MEM2 also uses the FMD-index for seeding, but uses a lower compression factor in its implementation to reduce memory bandwidth requirements. In particular, the occurrence table used for performing range queries on the FMD-index is decompressed by x and the suffix array to identify locations of substrings in the reference genome is fully decompressed. These changes increase the FMD-index size to 42 GB (12 GB occurrence table+30 GB suffix array) compared to 4.3 GB in BWA-MEM.

Starting from a single character in the read, the FMD-index enables forward and backward MEM searches to determine the number of hits of progressively longer substrings using at most two extra memory lookups per character. However, these memory lookups touch different parts of a 42 GB data structure and rarely exhibit spatial locality. This reduces the effectiveness of caching in modem processors and leads to high memory bandwidth requirements. Experiments on real whole human genome reads show that each read can require ˜61.3 KB of index data for seeding. In this disclosure, several techniques are proposed to improve the spatial and temporal locality of seeding and reduce the data requirements to ˜14 KB per read.

FMD-Index based seeding also inherently involves sequential dependent memory accesses and its performance is limited by memory access latency. This problem can be mitigated using hardware multiplexing, where one physical compute unit context switches between different reads on a memory stall.

FMD-index stores a compressed representation of the set of all suffixes that exist in the reference genome in lexicographical order. Consider a substring of length k in the read (referred to as a k-mer). Due to natural genome variation and machine read error, not all k-mers will exist in the reference and, hence, in the FMD-index. Therefore, when looking up a k-mer in the FMD-index, one must start with a 1-mer and grow the string, character by character, for as long as it exists in the FMD-index, or till one reaches the desired k-mer length. This iterative, character-by-character access to the FMD-index substantially increases the required number of DRAM accesses, creating a memory bottleneck. This is further aggravated by the fact that accesses to the index rarely follow lexicographic order, making it difficult to exploit locality over such a large window (i.e., set of all suffixes of the k-mer).

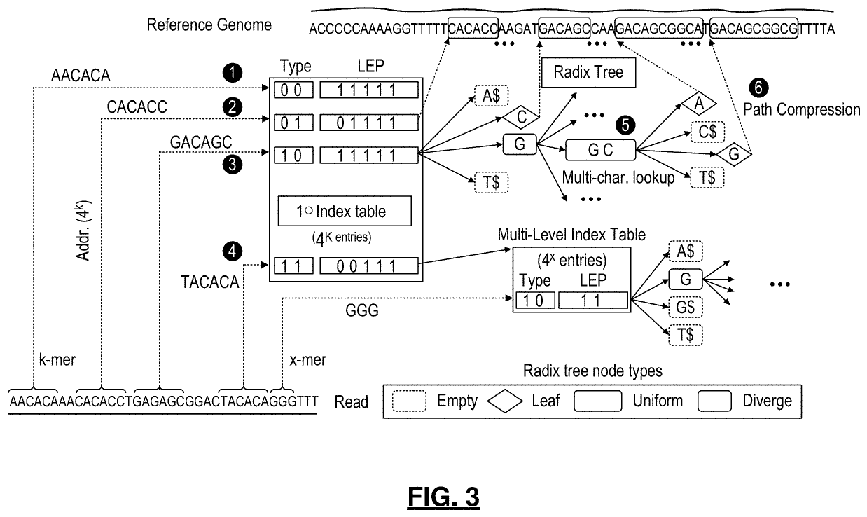

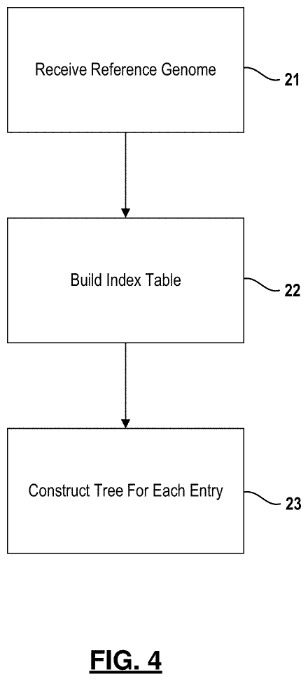

To overcome these two limitations, this disclosure enumerates all possible k-mers (whether they exist in the reference or not) and stores them in an index table. For each k-mer (an index entry), also store all its suffixes in the reference. Since all possible k-mers are represented in the index, k characters from the read can be looked up in a single memory access, significantly reducing the number of DRAM accesses. Furthermore, subsequent accesses to the suffixes of the k-mer have much improved spatial locality, since they are co-located together. FIG. 3 shows an example index table enumerating all 6-character substrings.

To choose k, one observes that BWA-MEM2 only reports SMEMs greater than a certain minimum length (e.g., 19). This is because shorter substrings lead to an excessive number of hits to be verified by seed extension. Thus, k can be set to any value less than 19. The higher k is set, the more characters can be looked up at once, but it would require more space. In one implementation, choose k=15 to keep the size of index table tractable (O(4k)), i.e., 1 G entries when k=15.

The next question is how to store the suffixes of a k-mer in an index entry, so that one can support MEM searches for strings longer than k. One option is to augment the index table with an FMD-index, and iteratively grow the k-mer prefix. However, even within the subset of all suffixes sharing the same k-mer prefix, FMD-index lookups have poor locality. Also, they still operate with a single character at a time.

To overcome this problem, one can observe that a radix tree can naturally support multi-character lookups. This is because in a radix tree, one can merge all singleton paths into a single node, thereby addressing a multiple character lookup with a single memory access. FIG. 3 shows a radix tree for one k-mer in the index table (note radix is 4 for the genome alphabet) The proposed seeding data structure (also referred to herein as ERT) merges singleton paths (GC in FIG. 3) using variable-size internal nodes that store the full singleton path string (designated as UNIFORM). A singleton path is encountered when all paths in the tree from a certain node onward share a common string.

To further improve the space-efficiency of the seeding data structure, one observes that a k-mer frequently becomes unique in the reference genome as it increases in length. This means that, past a certain length, a prefix is followed by a single, unique suffix string in the reference genome. This would introduce a UNIFORM node in the seeding data structure with a singleton string of characters (up to the length of the read). To avoid storing this long string, one instead replaces it with a pointer to the occurrence of this string in the reference genome. In FIG. 3, it is shown how in the seeding data structure, these nodes are marked as leaf nodes, containing a single pointer. Leaf nodes encountered during a MEM search are decompressed, by fetching the full reference string corresponding to the reference pointer stored at the leaf node. Note that the pointer in the leaf node is required regardless of this compression technique since it is necessary to indicate the location of the traversed k-mer in the reference genome. Hence, it does not present any storage overhead. Instead, this optimization results in ˜2× space savings and was critical for being able to store the full human genome in under 64 GB of storage, which is a common configuration for servers.

The k-mer index table and corresponding radix trees are built by first enumerating all possible k-mers and then exhaustively traversing the reference genome for each k-mer and growing the trees according to all existing sequences in the reference. Each k-mer and ERT path corresponds to a unique sequence in the reference. The locations of these sequences are stored as pointers at the leaves of the tree, as noted above. Note that if a particular k-mer does not exist (referred to as EMPTY in FIG. 3), one does not store a pointer to a tree since no SMEM k<19 is required. In an example implementation where k=15, approximately 38% of the index entries are empty. For an empty entry, one still compute its LEPs and store it in the index table to indicate at which positions along with k-mer a backward traversal must be initiated.

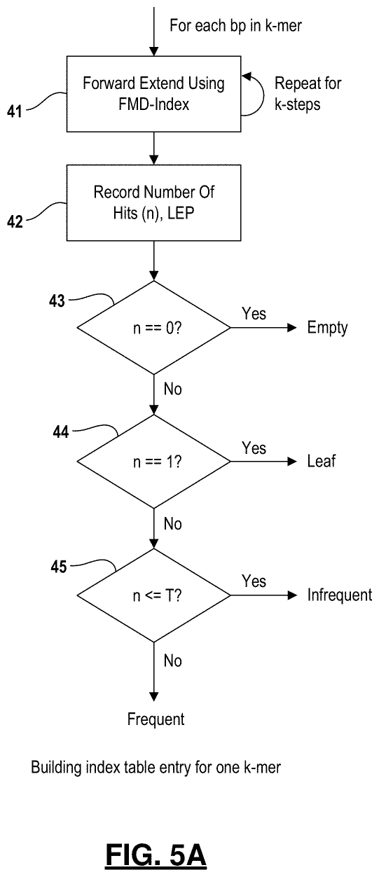

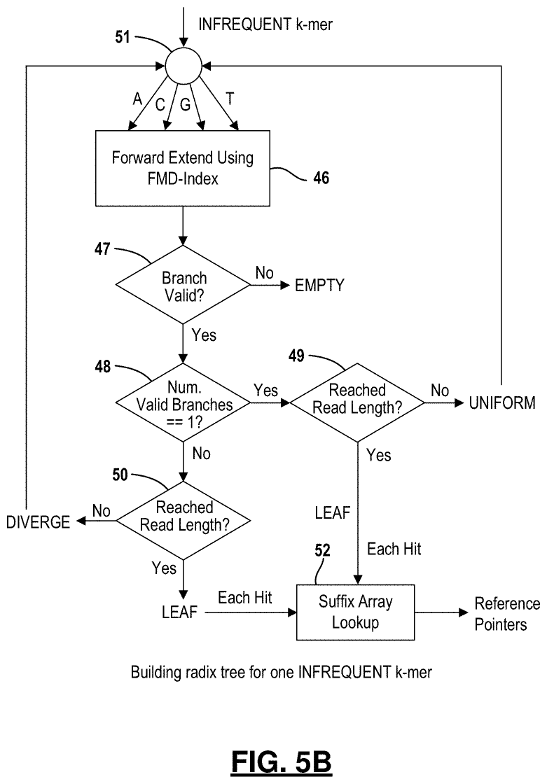

FIG. 4 further illustrates this method for constructing the seeding data structure. In one example, the seeding data structure is used to align reads to a genome sequence. As a starting point, the reference genome sequence is received at 21 by a computer processor. While reference is made aligning reads to a genome sequence, it is understood that the broader aspects of this disclosure are applicable to identifying matches for any type of character strings.

Next, an index table is built at 22 for the reference genome sequence in a computer memory. Each entry in the index table represents a k character string (or k-mer) in the reference genome sequence, where the k-mer is comprised of nucleotides. Additionally, the index table contains entries for all possible permutations for the k-mer.

In one example embodiment, the index table is built by generating permutations of the k character string; for each permutation, applying a hash function to a given permutation to form a hash value; and creating an entry for the permutation in the index table, such that the hash value corresponds to location of the entry in the memory. Each entry in the index table includes a presence indicator that indicates if the k character string exists in the reference genome sequence and the pointer to the tree in the secondary data structure. Building the index table may further include searching for a given entry in the reference genome sequence and labeling the given entry as empty in the index table if the given entry is not found in the reference genome sequence.

For each entry in the index table, a radix tree for a given entry of the index table is constructed at 23 in a secondary data structure of the computer memory, such that the given entry of the index table includes a pointer to the tree in the secondary data structure and the tree represents suffixes to the given entry as found in the reference genome sequence.

More specifically, a radix tree is constructed by a) appending a possible value for a character to a previous string to form a new string; b) determining a number of occurrence of the new string in the reference genome sequence; c) adding a branch to the tree when the number of occurrences of the new string in the reference genome sequence is more than zero; and d) setting the previous string equal to the new string, where an initial state of the previous string is the permutation of the k character string represented by the given entry and steps a)-d) are performed for each possible value of the characters comprising the reference genome sequence. Multiple branches are added to the tree when the number of occurrences of the new string is more than zero for two or more of the possible values for the characters in the reference genome sequence, such that each of the multiple branches terminates at a node and the node includes a pointer to another node of the tree. This process is repeated until only one occurrence of the new string is found in the reference genome sequence across each of the possible values for the characters in the reference genome sequence or the new string has the same suffix (with length=read length−k) at all its occurrences in the reference genome.

Returning to FIG. 3, the proposed seeding data structure is described in more detail. The proposed seeding data structure 30 uses a combination of an index table (or k-mer table) 31 for fast exact matching of k-mers, and a variant of a radix tree 32, to create variable length seeds from these k-mers. Each entry is the index table is a k character string (e.g., 15 characters). A suitable value of k is chosen for the index table after considering the sparsity of occurrence of the k-mer in the reference genome and the additional metadata overhead required to uniquely identify the k-mer.

In the example embodiment, each entry in the index table includes: (1) two bits to indicate the type of the index table entry; (2) a (k−1) bit LEP vector, indicating positions in the read where the set of candidate reference locations change; and (3) a pointer to the root node of the radix tree, with the k-mer as prefix. Values for the type of index table entry may include but are not limited to: 00 indicates an EMPTY entry (i.e., k-mer not found in the reference genome sequence) as indicated at 33; 01 indicates a LEAF entry as indicated at 34; 10 indicates an INFREQUENT entry, i.e., # hits for k-mer is less than or equal to a threshold T as indicated at 35; and 11 indicates a FREQUENT entry, i.e., # hits for k-mer is greater than a threshold T as indicated at 36. For this case, an additional x-mer from the read is used to lookup a second-level index table (an x-mer table). The threshold T may be 256 hits although other values are contemplated by this disclosure.

The radix tree contains entries for both internal nodes and leaf nodes. Leaf nodes in the radix tree include two fields: (1) count containing the number of times the seed occurs in the reference genome and (2) a pointer to the value of the k-mer in the reference genome sequence as indicated at 37.

For a genome sequence, each internal node can have up to four valid children, i.e., A, C, G or T. Different types of children nodes are indicated by a code. In one embodiment, the code is an eight bit binary number, where two bits are used to indicate the type of each of the children (i.e., A, C, G or T) of the node, respectively.

INFREQUENT entries in the index table may link to either DIVERGE internal nodes or UNIFORM internal nodes. DIVERGE internal nodes have more than one branch path. Fields for the DIVERGE internal nodes include: (1) a code representing the types of the children which are branched to, and (2) a set of pointers to the subtrees of each child of the internal node. On the other hand, UNIFORM internal nodes represent multiple occurrences in the reference genome but have only one child path. In additional to the code, the UNIFORM nodes store a string representing the base pairs (BPs) encountered along the single branch path, where the string is represented as a tuple (BP count, BPs).

Lastly, a secondary index table 40 exists for each FREQUENT entry 36 in the primary index table. Entries in this secondary index table are similar to the primary index table and the secondary index table (x-mer table) is similar in structure to the primary index table.

FIGS. 4A and 4B illustrates methods for constructing the index table 31 and the radix tree 32, respectively. This data structure is built offline once for each reference genome. The same index can be reused for processing several whole-genome samples containing billions of reads.

To populate each index table entry, prefixes of the k-mer (starting with length 1 up to k) are extended by performing forward search on the FMD-index as indicated at 41 of FIG. 4A. During forward search, record positions in the read at 42, where the set of candidate hits changes and construct the left extension point (LEP) list. After repeating the forward extend step for up to k steps, the number of hits is determined for each k-mer. The number of hits is used to indicate the index table entry type. If the number of hits is zero, the entry type is set to EMPTY as indicated at 43 and processing continues as described in relation to FIG. 4B. If the number of hits is one, the entry type is set to LEAF as indicated at 44 and processing continues as described in relation to FIG. 4B. If the number of hits is less that the threshold T, the entry type is set to INFREQUENT as indicated at 43 and processing continues as described in relation to FIG. 4B. If the number of hits is greater than the threshold T, the entry type is set to FREQUENT. For FREQUENT k-mers, a secondary index (x-mer) table is built by performing x steps of the forward extension as described above.

Next, given an infrequent k-mer (represented by the root node of the radix tree), forward extend the k-mer by each of the four possible base pairs (A, G, C and T) and build a radix tree for the k-mer as seen in FIG. 4B. Each path from the root node to any internal/leaf node represents a prefix of the suffix of the reference genome.

If the result of forward extension indicates no valid branches, record an EMPTY entry in the CODE field for the branch as indicated at 47. If there are more than one valid branches for the node, a DIVERGE entry is used as indicated at 48. If the depth of the child node is equal to the read-length, a LEAF type entry is used as indicated at 50; otherwise, processing continues at 51. In case there is a single valid branch, record an UNIFORM entry and keep track of the number of base pairs observed along the single branch path and processing continues as indicated at 49. On the other hand, if the single branch-path extends up to read-length base pairs, store a LEAF entry instead. A suffix array lookup is used at 52 to identify the reference genome locations containing the string represented by the LEAF node.

The proposed data structure is particularly suitable for read alignment of genome data and other string matching methods. For example, read alignment may be performed using the SMEM algorithm available in the Broad Institute's BWA-MEM software. The conventional SMEM algorithm relies on single character lookups using the FMD-index for forward and backward search. The proposed data structure by virtue of using an index table and radix tree can support multi-character lookups and improves locality of the SMEM algorithm. Furthermore, it is observed that there are several redundant backward searches in the conventional algorithm, which can be pruned away by performing backward searches in a right-to-left order. Augmenting prefix information at the leaf nodes also enables us to skip certain redundant backward searches compared to the original algorithm.

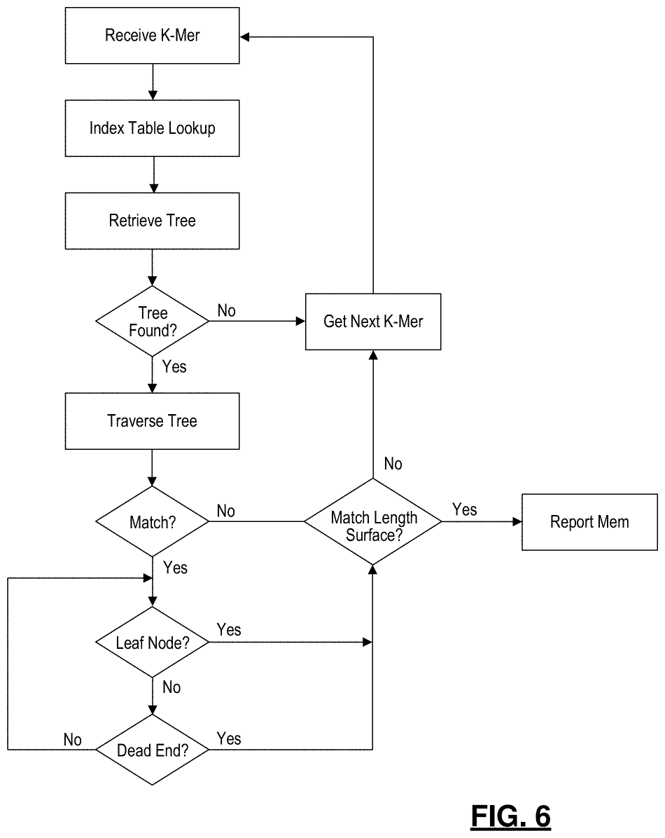

Once constructed, the seeding data structure 30 can be used to search for maximal exact matches (MEMs) according to the SMEM seeding algorithm. An example method for identifying matches between strings in a read and a portion of a reference genome sequence using the seeding data structure is further described in relation to FIG. 6. Upon receiving a k-mer from a read at 61, an entry corresponding to the k-mer is retrieved at 62 from the index table, for example using a single DRAM access. It is understood that the read is extracted from a biological sample from a subject.

Next, a tree for the k-mer is retrieved at 63 from the secondary data structure using the pointer in the retrieved entry from the index table. As described above, the tree represents suffixes to the entry as found in the reference genome sequence. In the event that no tree is found for the k-mer, another k-mer is retrieved from the read and processing continues as indicated at 70.

Branches of the retrieved tree are traversed at 65 to identify matches between strings in the read and strings found in the reference genome sequence. More specifically, branches of the tree are traversed by comparing characters in the read that follow the k-mer to suffixes represented by the tree. Branches of the tree continue to be traversed until a leaf node is encountered or a dead end is reached (i.e., no further characters in the read match with strings found in the reference genome sequence).

Upon encountering a leaf node at 66, the maximal exact match is reported as indicated at 68. To do so, at least a portion of the reference genome sequence is retrieved using the pointer in the leaf node and the characters in the read are compared to corresponding characters in the reference genome sequence to find the entirety of the matched strings which form the MEM. After reporting MEM, processing continues with another K-mer as indicated at 70.

In the example embodiment, only biologically significant strings are reported as MEMs. Therefore, the number of characters in the matched strings is compared to a threshold at 67 and only matched strings which exceed the threshold are reported as MEMs. In one example, k=15 and the threshold is 19. In the event that the number of characters in the matched strings does not exceed the threshold, the matched string is not reported and processing continues as indicated at 70.

Alternatively, traversing the tree may reach a given node in the tree where characters in the read do not match characters in the branches extending from given node (i.e., a dead end) as indicated at 69. Again, if the number of characters in the matched strings does not exceed the threshold (e.g., 19), the matched string is not biologically significant and processing continues as indicated at 70. On the other hand, if the number of characters in the matched string (equals or) exceeds the threshold, the end of a MEM has been identified. For reporting, all locations where this MEM exists in the reference genome sequence (i.e., all leaf nodes in the downstream sub-tree) are gathered using a depth-first traversal, referred to as leaf gathering. That is, leaf nodes downstream from the given node are retrieved; and for each leaf node, matched strings are reported as MEMs, including the locations of maximal exact match as found in the reference genome sequence.

Each time the path in the seeding data structure traverses a node with divergence, an LEP is marked since the divergence indicates that the number of hits is divided across the divergent paths from that node and is decreasing. After the depth first search reaches its dead-end (or the end of the read), a backward traversal is instigated for each LEP position along the traversed path. The backward traversal operates in the same way as the forward path and uses the same ERT data structure by searching for the reverse complement strings. Note that base-pairs A and T and base-pairs C and G are complements of each other.

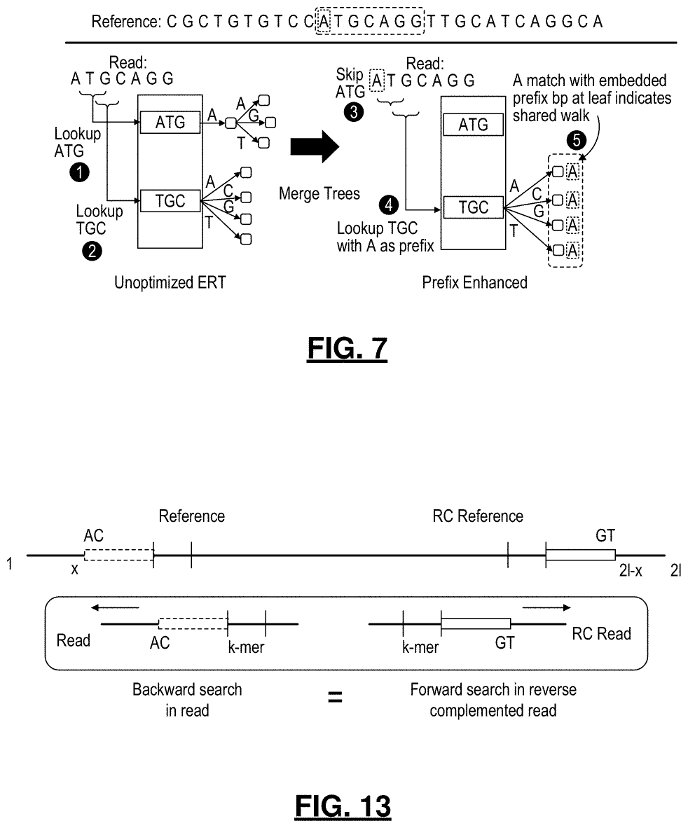

A few optimizations to this method are described below. The goal of prefix-merged radix trees is to re-use work across MEM searches from consecutive positions in the read. In the seeding computation, the time spent doing backward MEM searches is ˜2× that of forward search making it important to optimize this step. On average, one finds that there are ˜10 backward searches for each forward search from a pivot. Also it is common to observe backward searches from adjacent query positions in the read (consecutive bits of LEP are ‘1’). Normally, these lead to multiple independent index table lookups and tree traversals as shown in FIG. 7.

In the unoptimized seeding data structure 30, there exists a radix tree for each k-mer that occurs in the reference, including adjacent, sliding window k-mers (e.g., ATG and TGC). Radix trees for adjacent k-mers are recognized to contain redundant information and that the information contained in one of the trees can be reconstructed from the adjacent k-mer's tree by storing prefix information at each of its nodes. In the example shown in FIG. 7, a string ATGC, which is normally found by accessing the ATG tree can be instead reconstructed from the TGC tree by indicating the presence or absence of prefix character A in each of the nodes of TGC's tree.

The key observation is that with such a prefix-merged radix tree, multiple backward searches (TGCxyz and ATGCxyz) can be performed in a single index table lookup and tree traversal by checking for prefix character matches at each visited node. In FIG. 7, when the leaf node represented by string TGCA is reached, one can also match character A from the read as prefix, resulting in the MEM represented as ATGCA. This reduces two backward extensions into one.

Augmenting each of the nodes with prefix information in order to merge k-mer trees takes up significant space and offsets the benefit from merging trees. Therefore, in the prefix optimized seeding data structure, only leaf nodes are augmented with prefix characters (2 bits per prefix character) found at the corresponding reference positions (FIG. 7). Storing prefix information at the leaf nodes is sufficient as prefix information at each of the internal nodes can be reconstructed by visiting all of the leaf nodes in its corresponding sub-tree. If any of the leaf nodes of an internal node's sub-tree contains the desired prefix character, then the internal node also contains the prefix character. While storing prefix information at internal nodes does have the benefit of terminating some backward searches early in case of prefix mismatch, the space overhead outweighed the performance benefits was found.

Another design choice to be made for prefix-merged seeding data structure is the choice of prefix length. Each backward search on average matches ˜1 prefix character at the leaf nodes was observed, resulting in 50% fewer backward searches. As a result the seeding data structure supports 1-character prefix at leaf nodes. Although the above discusses the benefits of prefix-merged radix trees in the context of backward searches, it must be noted that forward MEM searches can also benefit from this optimization when initiated from adjacent positions in the read.

The goal of locality with k-mer reuse is to increase the re-use for the radix tree of a k-mer. Given the highly redundant nature of the human genome and high coverage of sequenced reads (each position in the reference genome can be covered by 30-50 reads on average), a few unique k-mers tend to be reused frequently in a batch of reads was observed. Ideally, we would like to fetch the radix tree for these k-mers only once to save memory bandwidth. Unfortunately several radix trees need to be accessed to find seeds for a read, and their aggregate size exceeds that of on-chip caches. As a result, a radix tree usually gets evicted before it can be reused by another read. This problem can be mitigated only if determined in advance the set of all k-mers for which a radix tree needs to be fetched from DRAM.

The forward and backward search phases of the SMEM algorithm can be decoupled to expose temporal locality. More specifically, forward search for a batch of reads can be performed, identify all the unique k-mers that are to be used in backward search (using LEPs), fetch each radix tree once for each unique k-mer and perform all backward searches for that k-mer tree before moving to the next k-mer. This technique is referred to as k-mer reuse.

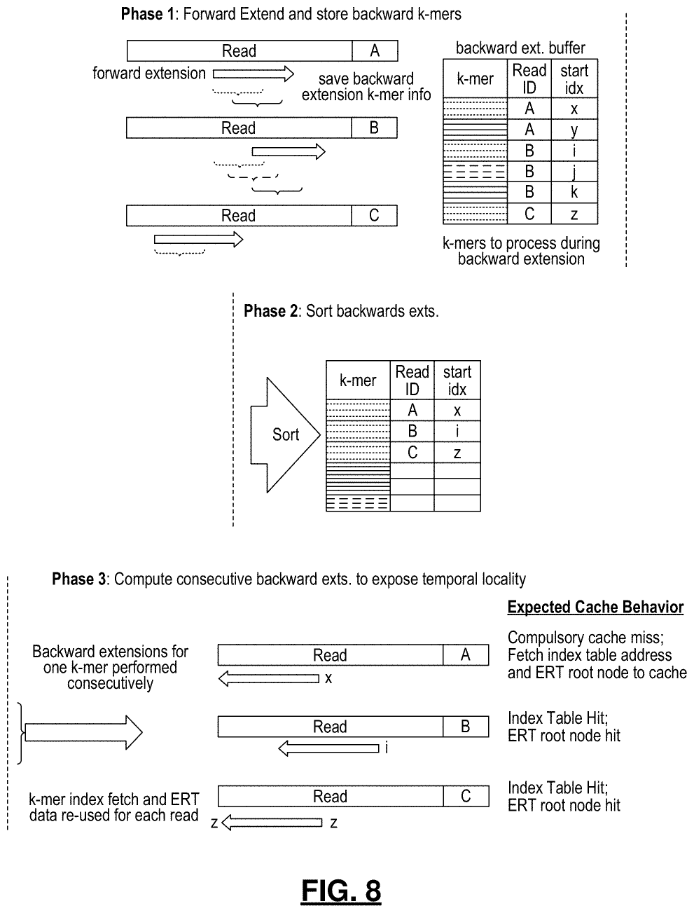

FIG. 8 describes the steps to be performed to leverage k-mer reuse. While processing the forward extensions for a batch of N reads, each backward extension that must be computed is stored in a k-mer metadata table implemented on-chip. Each backward extension entry is composed of: (1) k-mer starting from the backward extension point in the read, (2) the read ID in the batch, and (3) start position of backward extension in the read. Once all forward extensions have been completed for a batch of reads, all entries are sorted in the on-chip memory, grouping each required backward extension by k-mer. Then proceed one k-mer at a time and compute all backward extensions associated with a k-mer sequentially. The first time a k-mer is encountered, one index table lookup is performed, as well as fetch of portion of the k-mer's tree into an on-chip cache. Subsequent backward extensions then consult this cache during tree walking, skipping two otherwise mandatory DRAM accesses. If a backward extension needs data that does not exist in the tree cache, fetch it from memory on-demand and store it in case future backward extensions require this data. K-mer reuse strictly decreases the number of radix trees fetched from DRAM—and reduces total bandwidth requirement—but adds the computational overhead of sorting the backward extensions by k-mer.

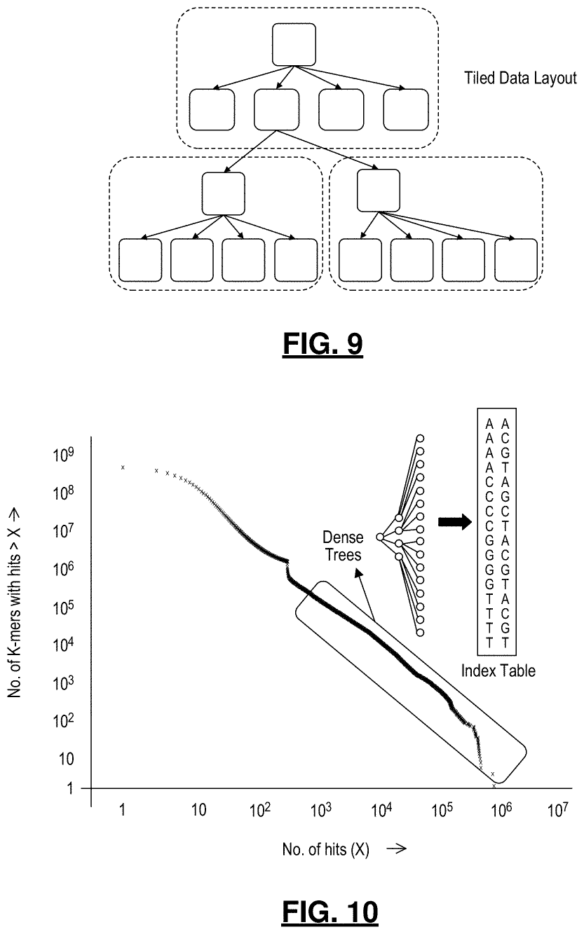

With reference to FIG. 9, a tiled layout for the nodes of the radix tree is adopted to improve spatial locality of accesses. In this layout, subtrees of nodes that are likely to be accessed at the same time are clustered together into a single cache block- or a DRAM page-sized tile. Compared to breadth-first or depth-first layout of nodes, the tiled layout guarantees at least log₄(n+1) nodes accesses per tile, where n is the number of nodes in the tile. With this optimization, the seeding data structure traverses ˜3 nodes on average per 64 B, utilizing 50% of the data it fetches from memory.

Enumerating all k-character prefixes in the index table can have prohibitive space overheads for large k. For example, 19-mer table has 4¹⁹ entries, resulting in 2 TB of space, assuming 8 bytes per entry. However, the human genome is not a random string of characters from the genome alphabet. The repetitive nature of the human genome makes the distribution of hits (or leaf nodes in the radix tree) for different k-mers heavily skewed.

The skewed distribution of k-mers in the human genome are leveraged to design a multi-level index table. For a given number of hits x, FIG. 10 shows the number of k-mers in the human genome that have hits >X. It can be seen that very few k-mers (˜0.01%) have greater than 1000 hits. However, these k-mers have dense radix trees, which can be compactly represented using an index table as shown in FIG. 10.

Instead of enumerating all k-character prefixes for large k, the index table is decomposed into two levels (FIG. 3), wherein the first level enumerates all k-mers and the subsequent level enumerates all x-character suffixes for a subset of k-mers (such that k+x=min. SMEM length). The multi-level index table further extends the benefit of multi-character lookup. Another way to visualize the multi-level index is as a high fan-out tree, with the root being the k-mer and the children being all x-character suffixes for the k-mer. While choosing a larger x helps reduce tree traversal time, for the human genome we were able to accommodate up to x=4 (fan-out=256) for a subset of 15-mer dense trees without increasing space overheads (only 0.35% of all 15-mers>100 leaf nodes). Compared to x=x=4 improves CPU performance by 10%. Since most trees are shallow (83% of leaf nodes have depths <=8), more than two-levels or high fan-out for internal nodes of the seeding data structure were not explored.

Typically backward search is performed starting from each query position where the set of candidate hits changes (as given by the LEPs), in no particular order. However by imposing an order for the backward extension pass, namely starting from the rightmost query position where the hit set changes and proceeding leftward, it is possible to prune out subsequent backward searches as illustrated in FIG. 11.

The forward pass partitions the read into multiple non-overlapping MEMs. As a result, each backward search is guaranteed to not produce a MEM that spans across multiple pivots. If any backward extension from position xⱼ in the read reaches the previous pivot xᵢ₋₁, then backward extensions ∀x, where x<xⱼ are guaranteed to produce MEMs that are contained within that of xⱼ and are redundant.

For exhaustive identification of all the SMEMs in the read, the forward search procedure must be repeated starting from every position in the read. This is wasteful and can lead to redundant computation. However, by supporting backward search in the same index, begin seeding only from those read positions at which hit sets changes have been recorded during forward search. To support backward search, make the observation that the two strands of DNA in the human genome are reverse complements of each other. Since we are unsure if the read originated from the forward or reverse strand, index both strands in the same index. This means that as shown in FIG. 13, backward search for a pattern from the read can be emulated by using forward search of reverse complemented pattern in the reverse complemented read. This is similar in principle to the FMD-index used in BWA-MEM. An alternative strategy is to build two separate indexes, one each for the forward and reverse complemented strands, however, this approach requires two lookups per k-mer. In space constrained scenarios, where only one of the strands can be indexed, SMEMs can be identified at the cost of doubling the number of index lookups. This is because both the forward and reverse complements of the k-mer have to be looked up in the index. Backward search can be supported by doing forward search on the reverse complemented read as before.

Seeding accelerator is described that has been designed to take full advantage of the data efficiency benefits provided by seeding data structure. The seeding accelerator leverages fine-grained context switching to hide the long latency of memory accesses and includes customized datapath and functional units to exploit re-use opportunities present in the seeding algorithm.

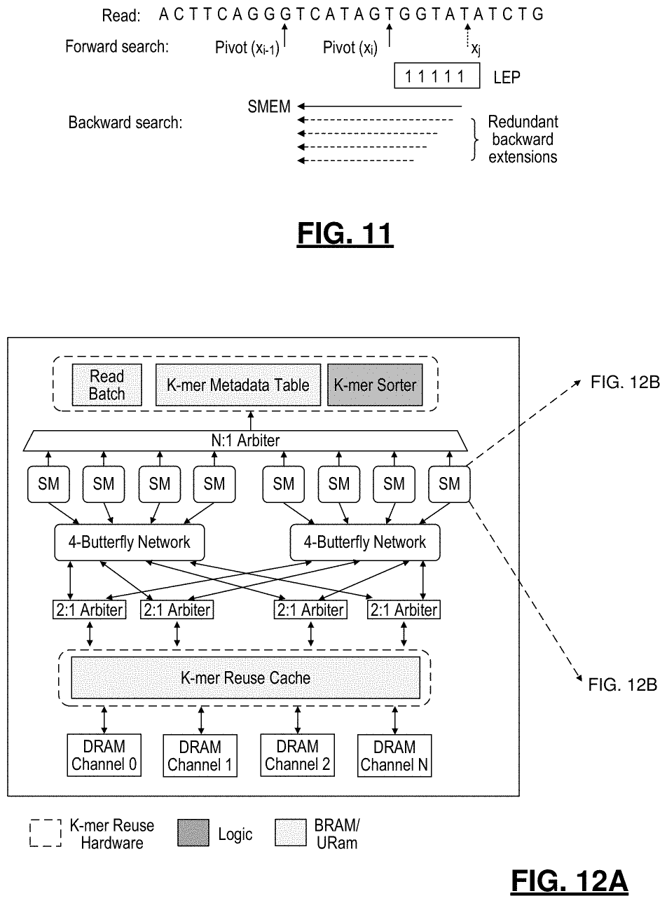

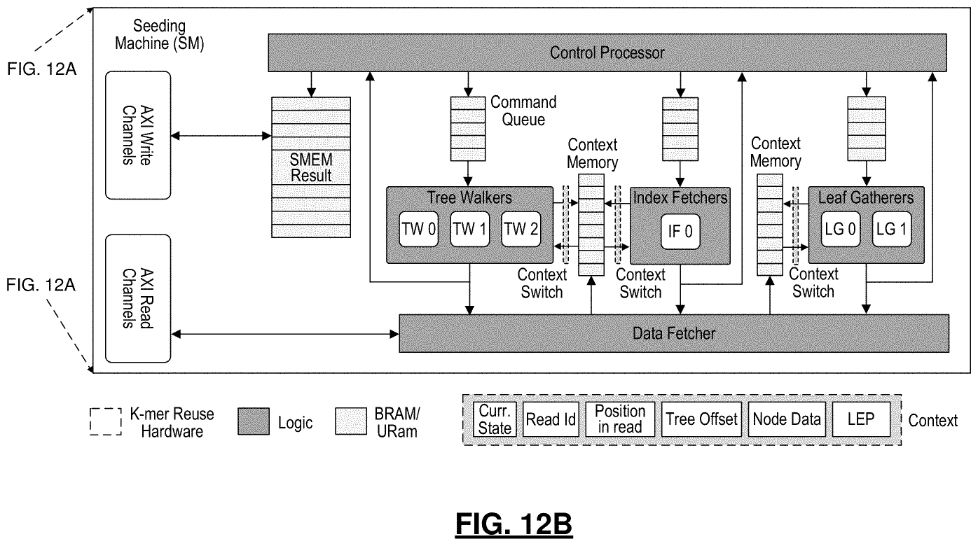

An example seeding processor architecture is shown in FIGS. 12A and 12B. The processor is composed of multiple parallel seeding machines connected to the available DRAM channels using a crossbar network. Each seeding processor is composed of a control processor that issues commands to three types of processing elements. Each processing element performs a sub-task associated with SMEM identification (i.e. index table lookups, walking radix trees, and depth-first leaf gathering). When a processing element issues a memory request to the Data Fetcher—a rudimentary address generation unit and memory controller—and a memory stall occurs, the processing element immediately switches to a new context. This context switching greatly increases compute density of each seeding machine and is essential to an FPGA implementation with limited logic and routing resources. When the memory request returns, its data is stored in the corresponding PEs context memory and the context is marked as ready.

The Index Fetcher is responsible for initiating a walk by converting a k-mer string to an index table address and requesting the corresponding entry from the ERT index table. These requests immediately trigger a context switch, swapping out the current context until the requested data is returned. The returned data indicates whether the k-mer exists in the reference, whether it is a singleton leaf path, or whether a corresponding radix tree exists that needs to be traversed. If the path terminates at the index table, the results are returned to the control processor to determine how to proceed. If the radix tree for that k-mer exists, the index fetcher issues a request for the root of the seeding data structure.

The Tree Walker is responsible for traversing the seeding data structure, decoding nodes, and reporting the end result of a walk. Each node in the tree is decoded using the corresponding base-pair in the read to calculate the next node address. If the Tree Walker ever detects that it needs more of the seeding data structure to continue its traversal, it requests the data from the Data Fetcher and triggers a context switch.

During decode, the Tree Walker computes the address of the next tree node based on the types and content of existing child nodes and the read characters or ends the traversal. Each radix tree node takes a variable number of cycles to decode depending on node complexity. For example, UNIFORM nodes require an exact match string comparison to compare each DNA base-pair in the uniform string with the read string. This comparison is accomplished using parallel XOR gates and priority encoders over three cycles. Leaf nodes that are compressed also require string comparison hardware. Implementing these comparisons using custom parallel hardware is an important feature of the specialized processor versus implementation in software on a general purpose CPU.

If a tree walk stops before reaching a leaf node, all remaining leaves in the tree must be gathered in order to identify all possible reference locations of the current MEM. This is referred to as Leaf Gathering, and is accomplished using depth-first search on the sub-tree. This depth-first search is accomplished by considering and decoding each base-pair (A,T,G,C) path in the radix tree and maintaining a stack of radix tree node indices that need to be explored. Nodes are decoded and traversed just as in the Tree Walker, however, the Leaf Gatherer does not need to perform string matching (required for early path compression and uniform nodes), and does not include string comparison hardware.

The control processor manages the high-level algorithm for SMEM search and issues commands to each processing element according to the results returned from each processing element and the current stage of computation. For example, if a forward walk finishes, the control processor looks at the start and end point, determines the condition of the finished walk, and issues a new command (e.g. get the leaves associated with the walk if the walk produced an SMEM, or start a new backward extension if the walk failed to produce an SMEM) to the corresponding processing element command queue. To simplify tree walking hardware, walker PEs do not have special hardware for forward or backward walks; the control processor issues a forward or backward walk command by providing a start index and the forward (for forward extensions) or reverse complemented read (for backward extensions). The control processor maintains a queue of pending tree walks to deal with variable tree traversal times and schedules walks from other reads to ensure good compute utilization.

The seeding accelerator provides enough flexibility to be repurposed for other bioinformatics algorithms that are based on the FM-index. For example, Centrifuge—a state-of-the-art metagenomic read classification algorithm—uses FM-Index-based MEM seeding on both the forward and reverse complemented input read strings. In order to implement Centrifuge's MEM algorithm using the seeding accelerator, one would only need to add new control FSMs to the Control Processor. AD other hardware structures (index fetchers, tree walkers, leaf gatherers, crossbar, and I/O) would remain untouched.

In order to perform k-mer reuse (FIG. 8), all backward extension LEPs for a forward MEM in a read must be exported to the k-mer metadata table. Backward extensions that share the same k-mer are grouped together using the parallel hardware sorter to group entries for the same k-mer (Phase 2 in FIG. 8). One can also implement a specially designed cache structure—the k-mer reuse cache—to cache index table lookups, root node accesses, and other seeding data structure accesses. For a group of backward k-mers (Phase 3 in FIG. 8), the first k-mer in a sorted group causes a compulsory miss for both the index table lookup and the root node access. However, each successive k-mer can be guaranteed to hit in the cache. Seeding data structure nodes, other than the root node, are cached but are not guaranteed to be re-used across path traversals. Because k-mer reuse forces the algorithm to generate MEMs out-of-order for a particular read, we must also store all MEMs for each read in intermediate on-chip storage, to perform MEM containment checks and finally produce SMEMs in a final reconciliation step.

The techniques described herein may be implemented by one or more computer programs executed by one or more processors. The computer programs include processor-executable instructions that are stored on a non-transitory tangible computer readable medium. The computer programs may also include stored data. Non-limiting examples of the non-transitory tangible computer readable medium are nonvolatile memory, magnetic storage, and optical storage.

Some portions of the above description present the techniques described herein in terms of algorithms and symbolic representations of operations on information. These algorithmic descriptions and representations are the means used by those skilled in the data processing arts to most effectively convey the substance of their work to others skilled in the art. These operations, while described functionally or logically, are understood to be implemented by computer programs. Furthermore, it has also proven convenient at times to refer to these arrangements of operations as modules or by functional names, without loss of generality.

Unless specifically stated otherwise as apparent from the above discussion, it is appreciated that throughout the description, discussions utilizing terms such as “processing” or “computing” or “calculating” or “determining” or “displaying” or the like, refer to the action and processes of a computer system, or similar electronic computing device, that manipulates and transforms data represented as physical (electronic) quantities within the computer system memories or registers or other such information storage, transmission or display devices.

Certain aspects of the described techniques include process steps and instructions described herein in the form of an algorithm. It should be noted that the described process steps and instructions could be embodied in software, firmware or hardware, and when embodied in software, could be downloaded to reside on and be operated from different platforms used by real time network operating systems.

The present disclosure also relates to an apparatus for performing the operations herein. This apparatus may be specially constructed for the required purposes, or it may comprise a computer selectively activated or reconfigured by a computer program stored on a computer readable medium that can be accessed by the computer. Such a computer program may be stored in a tangible computer readable storage medium, such as, but is not limited to, any type of disk including floppy disks, optical disks, CD-ROMs, magnetic-optical disks, read-only memories (ROMs), random access memories (RAMs), EPROMs, EEPROMs, magnetic or optical cards, application specific integrated circuits (ASICs), or any type of media suitable for storing electronic instructions, and each coupled to a computer system bus. Furthermore, the computers referred to in the specification may include a single processor or may be architectures employing multiple processor designs for increased computing capability.

The algorithms and operations presented herein are not inherently related to any particular computer or other apparatus. Various systems may also be used with programs in accordance with the teachings herein, or it may prove convenient to construct more specialized apparatuses to perform the required method steps. The required structure for a variety of these systems will be apparent to those of skill in the art, along with equivalent variations. In addition, the present disclosure is not described with reference to any particular programming language. It is appreciated that a variety of programming languages may be used to implement the teachings of the present disclosure as described herein.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A computer-implemented method for identifying a match between an input string and a portion of a reference genome sequence, comprising:
receiving, by a computer processor, a reference genome sequence;
building an index table for the reference genome sequence in a computer memory, where each entry in the index table represents a k-mer, the k-mer is comprised of nucleotides and the index table contains all possible permutations for the k-mer; and
for each entry in the index table, recording a presence indicator that indicates if the k-mer exists in the reference genome sequence; and
for each entry in the index table that exists in the reference genome sequence, constructing a tree for a given entry of the index table in a secondary data structure of the computer memory, where the given entry of the index table includes a pointer to the tree in the secondary data structure and the tree represents suffixes to the given entry as found in the reference genome sequence.

2. The method of claim 1 wherein building an index table further comprises
generating permutations of the k-mer;
for each permutation, applying a hash function to a given permutation to form a hash value; and
creating an entry for the permutation in the index table, such that the hash value corresponds to location of the entry in the memory.

3. The method of claim 1 wherein building an index table further comprises searching for a given entry in the reference genome sequence and labeling the given entry as empty in the index table if the given entry is not found in the reference genome sequence.

4. The method of claim 1 wherein each entry in the index table further includes a vector having k minus 1 elements, where each element in the vector corresponds to a subset of characters in the k-mer and the value of each element in the vector indicates whether a change occurred in the number of locations the subset of characters in the k-mer appears in the reference genome sequence.

5. The method of claim 1 wherein constructing a tree further comprises:
a) appending a possible value for a character to a previous string to form a new string;
b) determining a number of occurrence of the new string in the reference genome sequence;
c) adding a branch to the tree when the number of occurrences of the new string in the reference genome sequence is more than zero; and
d) setting the previous string equal to the new string
wherein an initial state of the previous string is the given entry and steps a)-d) are performed for each possible value of the characters comprising the reference genome sequence.

7. The method of claim 5 further comprises adding multiple branches to the tree when the number of occurrence of the new string is more than zero for two or more of the possible values for the characters in the reference genome sequence, where each of the multiple branches terminates at a node and the node includes a pointer to another node of the tree.

8. The method of claim 5 is repeated until only one occurrence of the new string is found in the reference genome sequence across each of the possible values for the characters in the reference genome sequence.

9. The method of claim 5 further comprises adding a leaf node to the tree when only one occurrence of the new string is found in the reference genome sequence across each of the possible values for the characters in the reference genome sequence, where the leaf node includes a pointer to the reference genome sequence.

10. The method of claim 1 further comprises extracting a read from a biological sample; and identifying matches in the between strings in the read and strings in the reference genome sequence using the index table and associated trees.

11. A computer-implemented method for identifying matches between strings in a read and a portion of a reference genome sequence, comprising:
extracting a read from a biological sample;
receiving, by a computer processor, a k-mer from the read;
retrieving an entry from an index table using the k-mer, where the index table contains an entry for each possible permutation of the k-mer and the entry includes a pointer to a tree in a secondary data structure;
retrieving the tree for the k-mer from the secondary data structure using the pointer, where the tree represents suffixes to the entry as found in the reference genome sequence;
traversing branches of the tree to identify matches between strings in the read and strings found in the reference genome sequence; and
reporting matches as maximal exact matches when number of characters in matched strings exceeds a threshold.

12. The method of claim 11 further comprises traversing branches of the tree by comparing characters in the read that follow the k-mer to suffixes represented by the tree.

13. The method of claim 11 wherein the tree was constructed by
a) appending a possible value for a character to a previous string to form a new string;
b) determining a number of occurrence of the new string in the reference genome sequence;
c) adding a branch to the tree when the number of occurrences of the new string in the reference genome sequence is more than zero; and
d) setting the previous string equal to the new string
wherein an initial state of the previous string is the entry from the index table and steps a)-d) are performed for each possible value of the characters comprising the reference genome sequence.

14. The method of claim 13 further comprises adding multiple branches to the tree when the number of occurrence of the new string is more than zero for two or more of the possible values for the characters in the reference genome sequence, where each of the multiple branches terminates at a node and the node includes a pointer to another node of the tree.

15. The method of claim 14 is repeated until only one occurrence of the new string is found in the reference genome sequence across each of the possible values for the characters in the reference genome sequence.

16. The method of claim 14 further comprises adding a leaf node to the tree when only one occurrence of the new string is found in the reference genome sequence across each of the possible values for the characters in the reference genome sequence, where the leaf node includes a pointer to the reference genome sequence.

17. The method of claim 16 wherein traversing branches of the tree include encountering a leaf node in the tree and, in response to encountering a leaf node and retrieving at least a portion of the reference genome sequence using the pointer in the leaf node, comparing characters in the read to corresponding characters in the reference genome sequence, and reporting a string with matched characters as a maximal exact match when number of characters in matched strings exceeds the threshold.

18. The method of claim 16 wherein traversing branches of the tree include encountering a given node in the tree where characters in the read do not match characters in the branches extending from given node and, in response to encountering the given node and when number of characters in matched strings exceeds the threshold, retrieving leaf nodes downstream from the given node, and reporting strings with matched characters as a maximal exact match, including locations of maximal exact match as found in the reference genome sequence.

19. The method of claim 13 further comprises retrieving another k-mer from the read when a tree for the k-mer is not found in the secondary data structure.

20. The method of claim 16 wherein each entry in the index table further includes a vector having k minus 1 elements, where each element in the vector corresponds to a subset of characters in the k-mer and the value of each element in the vector indicates whether a change occurred in the number of locations the subset of characters in the k-mer appears in the reference genome sequence such that in response to encountering a node in the tree with more than one branch, appending an element to the vector to indicate a change occurred the number of locations the matched string appears in the reference genome sequence.
